(19) United States
(12) Patent Application Publication
(10) Pub. No.: US 2022/0047454 A1
(43) Pub. Date: Feb. 17, 2022

(54) UTILITY CLUTCH FOR STIMULATING ACTIVITY AWARENESS FOR ABDOMINAL MUSCLE ENGAGEMENT

(71) Applicant: VALEH NAZEMOFF, SELBYVILLE, DE (US)

(72) Inventor: VALEH NAZEMOFF, SELBYVILLE, DE (US)

(21) Appl. No.: 17/355,132

(22) Filed: Jun. 22, 2021

Related U.S. Application Data
(60) Provisional application No. 63/065,569, filed on Aug. 14, 2020.

(51) Int. Cl. A61H 23/02

(52) U.S. Cl. CPC A61H 23/02; A61H 2201/5046; A61H 2205/083; A61H 2201/5084; A61H 2201/5097; A61H 2201/503

(57) ABSTRACT

An apparatus for stimulating activity awareness to engage abdominal muscles has a belt and buckle assembly designed to circumscribe a user's waist and fasten at the side. A front clutch with one or more containment members is horizontally coupled to an outside face of a vertically widened portion of the belt, facing outward. A back pouch is slidably coupled to the belt, a sealable back pouch containment member is designed to fold over the belt to be interior to an inside face of the belt, disposed against the user's lower back, and is able to contain cold/heat packs. One or more rechargeable batteries are disposed within the front clutch. A plurality of electrodes with accelerometers are disposed on an inside face of the vertically widened belt portion designed to vibrate upon detecting abdominal muscle movement, or lack of movement. A GPS and safety alert tracker may be included.

### CLAIM OF PRIORITY

This application claims priority to and the benefit of U.S. Provisional application with Ser. No. 63/065,569, filed on Aug. 14, 2020, with the title “Smart Wearable GPS Safety Alert Utility Clutch for Stimulating Activity Awareness for Abdominal Muscle Engagement”, which is incorporated herein by reference in its entirety.

### FIELD OF THE INVENTION

The inventive concept relates generally to a utility clutch for stimulating activity awareness to engage abdominal muscles.

### BACKGROUND

Currently, there are a number of wearable solutions for engaging the abdominal core muscles. One of these solutions attempts to stimulate and tone the abs for those too busy to exercise, stimulation through electrical muscle stimulators worn to contract the muscles and activate blood flow with vibration, but this solution fails to train the user, through active habits, to self-engage their abdominal core muscles to build strength, increase balance and stability, help prevent injuries, reduce back pain, and improve posture. Another wearable activity solution notifies and tracks movement, but this solution is unable to meet the need for focusing on notifying and tracking abdominal muscle engagement. Still another wearable solution seeks to utilize a core belt, but this solution fails to be worn over clothing and meet market needs because the belt does not store other items to make it a hands-free device with a GPS safety alert unit. Therefore, there currently exists a need in the market for an apparatus that stimulates awareness to the user to engage their abdominal core and carries items.

### SUMMARY OF THE INVENTION

The inventive concept includes a utility clutch for stimulating activity awareness to engage abdominal muscles that has a belt and buckle assembly with a belt member designed to circumscribe the user's waist and a buckle assembly designed to be adjustably fastened, so as to position the buckle assembly substantially to the right or left side of the user's waist. A front pouch assembly is horizontally coupled to an outside face of a vertically widened portion of the belt member, facing outward from the user, the front pouch assembly having one or more containment members. A back pouch assembly is slidably coupled to the belt member of the belt and buckle assembly, a sealable back pouch containment member designed to fold over the belt member so as to be interior to an inside face of the belt member and disposed substantially against the user's lower back, the back pouch containment member designed to contain one or more cold pack or heat pack members and include magnetic alignment. One or more rechargeable battery assemblies are disposed within the front pouch assembly. The front clutch assembly may have a recharging port. A plurality of electrode assemblies is disposed on an inside face of the vertically widened belt portion, electronically connected to the battery assembly, at least one accelerometer assembly disposed within each of the plurality of electrode assemblies, the electrode assemblies designed to vibrate upon detecting abdominal muscle movement, including lack of movement, to notify users to engage muscles. Electrodes record movement and may send data to apps that will help users be more aware of core muscle engagement and breathing.

Clutch herein is defined as a small bag or other flexible receptacle, typically carried in a pocket or attached to a belt.

In one embodiment of the utility clutch for stimulating activity awareness to engage abdominal muscles for a user, the one or more containment members are designed to be in an open, closed, or partially open state by slidably moving one or more tab members along two flexible strips of metal or plastic with interlocking projections often known as a zipper.

In one embodiment of the utility clutch for stimulating activity awareness to engage abdominal muscles for a user, the one or more containment members include a smartphone containment member and a pocket member containing a plurality of sealable or non-sealable compartments suitable for billfolds currency, credit cards and identification card storage, and other items.

In one embodiment of the utility clutch for stimulating activity awareness to engage abdominal muscles for a user, a plurality of loop members is outwardly disposed on an outside face of the belt member.

In one embodiment of the utility clutch for stimulating activity awareness to engage abdominal muscles for a user, a plurality of substantially planar magnet members is horizontally disposed on an inside face of the back pouch containment member, the magnet members substantially covered by a fabric portion of the back pouch containment member and designed to rest substantially against a user's lower back.

In one embodiment of the utility clutch for stimulating activity awareness to engage abdominal muscles for a user, a Bluetooth GPS and safety alert tracker is disposed on the front pouch assembly.

One embodiment of the utility clutch for stimulating activity awareness to engage abdominal muscles provides a smart wearable health and safety smart utility unit that stimulates user-awareness to engage the core abdominal muscles and practice proper breathing techniques by sending notifications through electrodes. Electrodes may motivate movement to boost the immune system for those sitting for long periods—behind a desk or table; on a couch, sofa, or chair; within vehicles such as cars, trucks, planes, or trains; or using other traveling methods. Engagement in the same region enhances respiratory muscles by applying breathing techniques. The utility clutch for stimulating activity awareness to engage abdominal muscles in this embodiment includes electrodes equipped with accelerometers that detect muscle movement. The electrodes will vibrate to notify the user to engage muscles. Electrodes record movement and may send various data to the app, which will help users to be more aware of core muscle engagement and breathing. Electrodes target various abdominal groups; obliques, transverse, and rectus abdominis muscles.

In some embodiments, a user can wear the utility clutch for stimulating activity awareness to engage abdominal muscles around his or her waist as if the utility clutch for stimulating activity awareness to engage abdominal muscles were a fashion accessory belt. Dual zipper pockets may be used for the containment members consisting of items such as a smartphone, money, card, currency, and wallet storage. Back pouch for icy/hot pack insertion for back pain also includes magnetic alignment support in this embodiment. The utility belt in some embodiments features attachments to hook D-rings to carry various items. A Bluetooth GPS safety alert Unit tracker is included. The utility clutch for stimulating activity awareness to engage abdominal muscles provides a stylish unisex hands-free design accessory to carry common everyday items; smartphone, currency, ID, cards, keys, and other items. In one embodiment, a dual zipper design provides easy and fast access to common items such as currency, credit cards, and ID card storage, and change compartments. The main compartment is designed with the user view in mind. Inside, the money storage view from the top makes it easy for ID and credit cards to be viewed, accessed, and removed. The currency storage area fits various sized currency and passports, making it ideal for travel. The change compartment also includes a zipper to prevent change from falling out.

One embodiment of the utility clutch for stimulating activity awareness to engage abdominal muscles includes a separate dual zippered smart device compartment for cell phones. In one embodiment, a dual zipper is designed for fast and easy access regardless of whether the user is right or left-handed. The dual zipper prevents content from falling out.

In one embodiment, a mobile phone can be charged, or an earphone can be connected, to the phone without taking the phone out of the smartphone container assembly providing a hands-free experience. In one embodiment, an add-on attachment capability is present for D-Ring hooks to carry various items such as hand sanitizers, keys, and flashlights.

Included may be: 1) Bluetooth GPS and safety alert unit, 2) Bluetooth GPS and safety alert tracker for seniors with Alzheimer's, dementia, those with autism, and wanderers, and 3) a safety alert button for those at high risk of falls or those needing help. In some embodiments, an inside layer includes a soft, breathable fabric. Clasp buckles may provide a comfortable material for the traveler, elder, and others, who fall asleep on their side wearing their smart utility pouch. In some embodiments, belt straps are adjustable from both sides to allow users to place front and back pouches at optimal positions.

The utility clutch for stimulating activity awareness to engage abdominal muscles may include a charging port with three functionalities: 1. Charges the internal Lithium-ion battery included in the compartment unit so that the phone can be charged while on the go. 2. Charges the Bluetooth GPS and safety alert Unit. 3. Charges the electrodes. The unit may include an ODM charger. Mobile applications of the utility clutch for stimulating activity awareness to engage abdominal muscles may have the option to connect to healthcare providers and care support to send and receive data from the electrode, for example, from physical therapists, caregivers, and fitness coaches. Such an app could include live or recorded video training for different types of exercises to motivate, for example, breathing and core strength building. The app's neuropsychology and cognitive behavior considerations encourage and motivate movement. Positive reinforcement elevates movement performance and helps users become more alert, aware, and engaged.

Some embodiments provide optional designs, including a rectangular or oval GPS/Alert unit button and squared or round attachment areas for the D-ring hooks. Optional materials in this embodiment include molded parts available in alternative softer material selections such as thermoplastic rubber.

In some embodiments of the utility clutch for stimulating activity awareness to engage abdominal muscles for a user, the rear containment pouch is designed to provide some back-pain relief for users who have been sitting too long or lack movement and core engagement. Included in the inside mesh lining of the pouch in this embodiment is a magnetic back alignment assembly used for pain therapy. Within the back pouch is space to insert a hot or cold pack. The back pouch can be used with pants that have a belt loop. The flap may also be height-adjustable through a power stretch mesh to target different areas of the back where relief is most needed.

The inventive concept may be made from natural materials such as cloth or leather, or it may be made from synthetic materials such as polymers.

An accelerometer is a tool that measures proper acceleration. Proper acceleration is the acceleration of a body in its own instantaneous rest frame; this is different from coordinate acceleration, which is acceleration in a fixed coordinate system.

It would be advantageous to have a utility clutch for stimulating activity awareness to engage abdominal muscles that is portable. Furthermore, it would be advantageous to have a utility clutch for stimulating activity awareness to engage abdominal muscles that is rechargeable. Still further, it would be advantageous to have a utility clutch for stimulating activity awareness to engage abdominal muscles that has compartments.

The inventive concept advantageously fills the aforementioned deficiencies by providing a utility clutch for stimulating activity awareness to engage abdominal muscles, which provides a way to engage a user's core and carry personal items.

The inventive concept fulfills the need for a wearable core activity notification and tracking device.

Among other things, it is an advantage of the utility clutch for stimulating activity awareness to engage abdominal muscles to provide a smart clutch that does not suffer from problems or deficiencies associated with prior solutions.

It is still further an advantage of the utility clutch for stimulating activity awareness to engage abdominal muscles to be rechargeable.

Still further, the utility clutch for stimulating activity awareness to engage abdominal muscles has an adjustable waist belt.

The inventive concept now will be described more fully hereinafter with reference to the accompanying drawings, which are intended to be read in conjunction with both this summary, the detailed description and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete, and will fully convey the full scope of the inventive concept to those skilled in the art.

### BRIEF DESCRIPTION OF THE DRAWINGS

FIG. 1 illustrates an exterior view of the utility clutch for stimulating activity awareness to engage abdominal muscles for a user.

FIG. 2 illustrates an inside view of the utility clutch for stimulating activity awareness to engage abdominal muscles for a user.

FIG. 3 illustrates a top exterior view of the back pouch of the utility clutch for stimulating activity awareness to engage abdominal muscles for a user.

FIG. 4 illustrates an inside view of the back pouch of the utility clutch for stimulating activity awareness to engage abdominal muscles for a user.

FIG. 5 illustrates a top view of the back pouch of the utility clutch for stimulating activity awareness to engage abdominal muscles for a user.

FIG. 6 illustrates the top front clutch view of the utility clutch for stimulating activity awareness to engage abdominal muscles for a user.

FIG. 7 illustrates a perspective view of the battery assembly and charging port.

FIG. 8 illustrates a perspective view of the pocket member containing a plurality of billfold assembly of currency, credit cards and identification card storage, and change compartment.

FIG. 9A-9B illustrates a method of using the utility clutch for stimulating activity awareness to engage abdominal muscles for a user.

FIG. 10 illustrates hot and cold packs for use in connection with the utility clutch.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

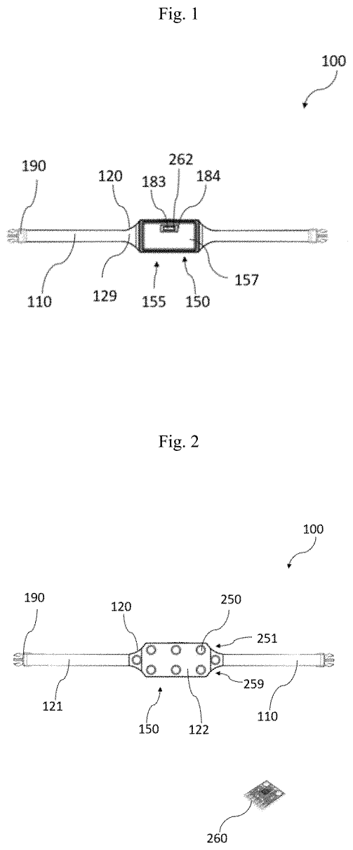

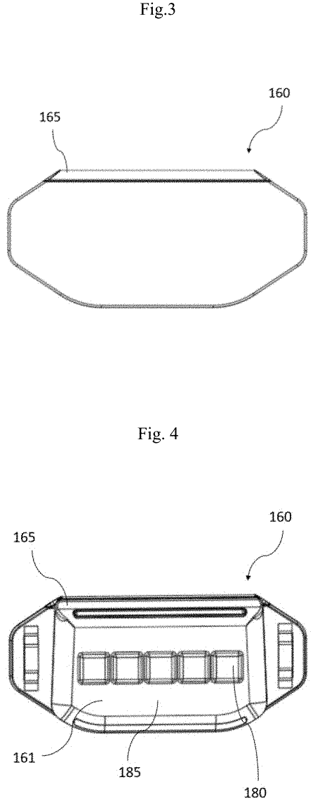

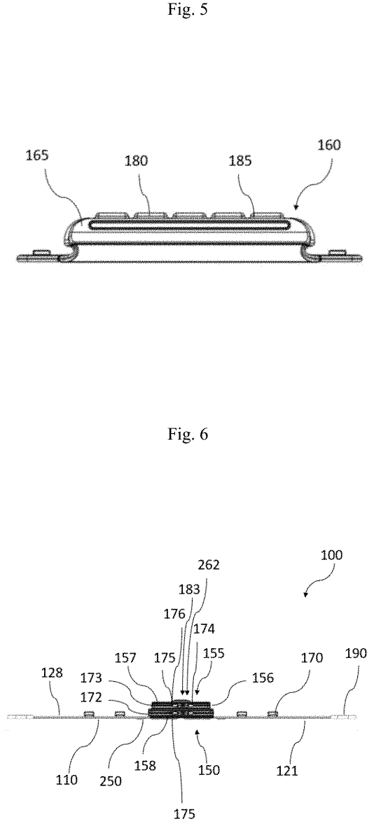

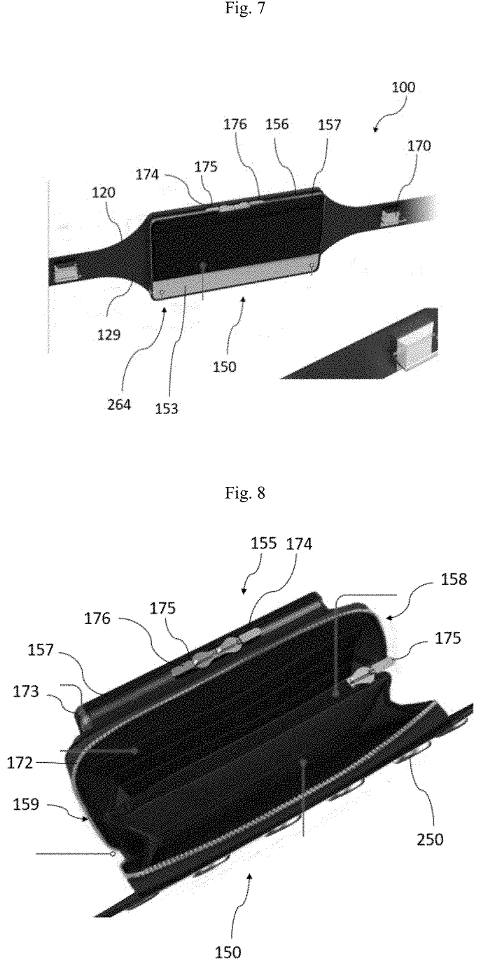

Referring to the Figures, FIGS. 1-8 illustrate an example embodiment for a utility clutch 10 for stimulating activity awareness to engage abdominal muscles for a user. The utility clutch 10 has a belt and buckle assembly 100 with a belt member 110 designed to circumscribe the user's waist and a buckle assembly 190 designed to be adjustably fastened so as to position the buckle assembly 190 substantially to the right or left side of the user's waist. A front clutch assembly 150 is horizontally coupled to an outside face 129 of a vertically widened portion 120 of the belt member 110, facing outward from the user, the front clutch assembly 150 having one or more containment members 155. A back pouch assembly 160 is slidably coupled to the belt member 110 of the belt and buckle assembly 100, a sealable back pouch containment member 165 is designed to fold over the belt member 110 so as to be interior to an inside face 121 of the belt member 110 and disposed substantially against the user's lower back, the back pouch containment member 165 designed to contain, with reference to FIG. 10, one or more cold pack members 166 or one or more heat pack members 167.

FIGS. 1-8 further illustrate the example embodiment wherein one or more rechargeable battery assemblies 153 are disposed within the front pouch assembly 150. A plurality of electrode assemblies 250 is disposed on an inside face 122 of the vertically widened belt portion 120, electronically connected to the battery assembly 153. In the example embodiment, at least six electrode assemblies 250 are disposed on the inside face 122 of the vertically widened belt portion 120 arranged so as to have at least one upper row of electrode assemblies 251 and at least one lower row of electrode assemblies 259. At least one accelerometer assembly 260 is disposed within each of the plurality of electrode assemblies 250, the electrode assemblies 250 designed to vibrate upon detecting abdominal muscle movement, including lack of movement, to notify users to engage muscles.

FIGS. 1-8 further illustrate the example embodiment of the utility clutch for stimulating activity awareness to engage abdominal muscles for a user wherein the one or more containment members 155 are designed to be in an open, closed, or partially open state by slidably moving one or more tab members 175 along two flexible strips of metal or plastic with interlocking projections 156 often known as a zipper. In this embodiment, the one or more containment members 155 are designed to be in an open, closed, or partially open state by slidably moving one or more of a left tab member 174 and a right tab member 176 along two flexible strips of metal or plastic with interlocking projections 156.

FIGS. 1-8 further illustrate the example embodiment of the utility clutch for stimulating activity awareness to engage abdominal muscles for a user wherein the one or more containment members 155 include a smartphone containment member 157 and a pocket member 158 containing a plurality of sealable or nonsealable compartments 159 for billfold assembly currency, credit cards and identification card storage, and other items.

FIG. 1-8 further illustrates the example embodiment of the utility clutch for stimulating activity awareness to engage abdominal muscles for a user wherein a plurality of D-ring or loop members 170 is outwardly disposed on an outside face of the belt member 128.

FIGS. 1-8 further illustrate the example embodiment of the utility clutch 10 for stimulating activity awareness to engage abdominal muscles for a user wherein a plurality of substantially planar magnet members 180 is horizontally disposed on an inside face 161 of the back pouch containment member 165, the magnet members 180 substantially covered by a fabric portion 185 of the back pouch containment member 165 and designed to rest substantially against a user's lower back.

FIGS. 1-8 further illustrate the example embodiment of the utility clutch 10 for stimulating activity awareness to engage abdominal muscles for a user wherein a Bluetooth GPS 183 and safety alert tracker 184 are disposed on the front clutch assembly 150. In the example embodiment, five or more substantially planar magnet members 180 are horizontally disposed on an inside face 161 of the back pouch containment member 165. The back pouch containment member 165 is further designed to contain the one or more hot pack members 166 or the one or more cold pack members 167.

FIGS. 1-8 further illustrate the example embodiment of the utility clutch 10 for stimulating activity awareness to engage abdominal muscles for a user wherein one or more electrode assemblies 250 are designed to record movement and send data to apps via the at least one or more of Bluetooth GPS 183, accelerometers 260, and at least one safety alert tracker 262.

FIGS. 1-8 further illustrate the example embodiment of the utility clutch 10 for stimulating activity awareness to engage abdominal muscles for a user wherein at least one charging port member 264 is disposed within the utility clutch electrically coupled to at least one or more of the rechargeable battery members 153, the Bluetooth GPS 183, and the at least one safety alert tracker 262.

FIGS. 1-8 further illustrates the example embodiment of the utility clutch 10 for stimulating activity awareness to engage abdominal muscles for a user that has an inner containment member 172 and an outer containment member 173, the inner containment member being wider and taller than the outer containment member 173 and having a plurality of the interior pocket members 158 therein. At least six of the electrode assemblies 250 are disposed on the inside face 122 of the vertically widened belt portion 120 arranged so as to have the at least one upper row 251 and the at least one lower row 259 of the electrode assemblies 250, the electrode assemblies 250 electronically connected to the rechargeable battery member 153, at least one accelerometer assembly 260 disposed within each of the at least six of the electrode assemblies 250, the electrode assemblies 250 designed to vibrate upon detecting abdominal muscle movement, including lack of movement.

FIGS. 1-8 further illustrates the example embodiment of the utility clutch 10 for stimulating activity awareness to engage abdominal muscles for a user wherein at least three of the pocket members 158 are disposed within the interior containment member 172, at least one of the at least three pocket members 158 sealable via the two flexible strips of metal or plastic with interlocking projections 156, the pocket members 158 further designed to be viewable from the top, and wherein the outer containment member 173 is further designed to contain a smartphone.

FIGS. 1-8 further illustrate the example embodiment of the utility clutch 10 for stimulating activity awareness to engage abdominal muscles for a user wherein the Bluetooth GPS 183 and at least one safety alert tracker 262 are disposed on the front clutch assembly 150.

FIGS. 1-8 further illustrate the example embodiment of the utility clutch 10 for stimulating activity awareness to engage abdominal muscles for a user wherein one or more of the electrode assemblies 250 are designed to record movement and send data to apps via at least one or more of the Bluetooth GPS 183, accelerometers 260, and Safety alert trackers 262.

In the example embodiment of the utility clutch for stimulating activity awareness to engage abdominal muscles for a user, the at least one charging port member 264 is disposed within the utility clutch electrically coupled to at least one or more of the rechargeable battery members 153, the Bluetooth GPS 183, and the at least one safety alert tracker 262, the rechargeable battery member 153 further being lithium-based.

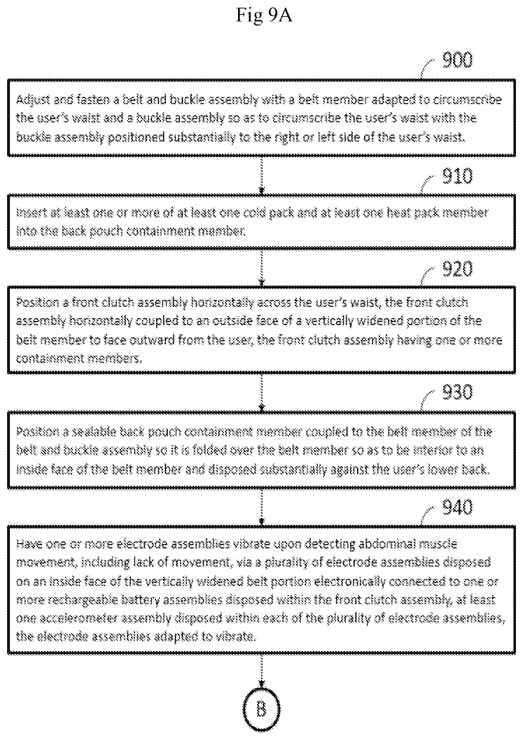

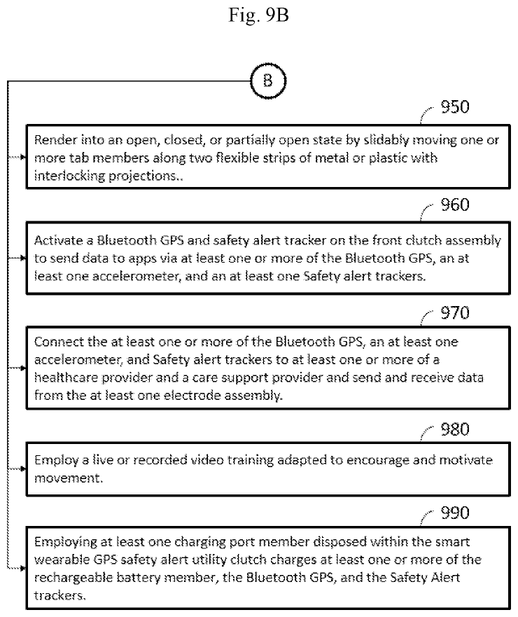

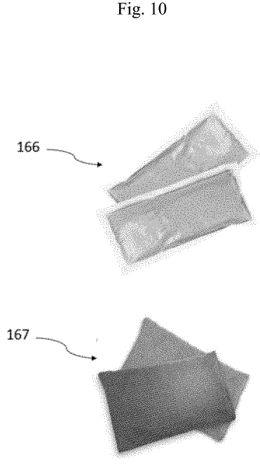

FIG. 9A illustrates an example method for wearing a utility clutch for stimulating activity awareness to engage abdominal muscles for a user. The exemplified method 900 includes adjusting and fastening the belt and buckle assembly 100 with the belt member 110 designed to circumscribe the user's waist and the buckle assembly 190 so as to circumscribe the user's waist with the buckle assembly 190 positioned substantially to the right or left side of the user's waist. The exemplified method includes 910 inserting at least one or more of at least one cold pack member 166 and at least one heat pack member 167 into the back pouch containment member 165. The exemplified method includes 920 further positioning the front clutch assembly 150 horizontally across the user's waist, the front clutch assembly 150 horizontally coupled to the outside face 129 of the vertically widened portion 120 of the belt member 110 to face outward from the user, the front clutch assembly 150 having one or more containment members 155. The example embodiment further includes 930 positioning the sealable back pouch containment member 165 coupled to the belt member 110 of the belt and buckle assembly 100 so it is folded over the belt member 110 so as to be interior to the inside face of the belt member 110 and disposed substantially against the user's lower back. This example embodiment further includes 940 having one or more electrode assemblies 250 vibrate upon detecting abdominal muscle movement, including lack of movement, via the plurality of electrode assemblies 250 disposed on the inside face 122 of the vertically widened belt portion 120 electronically connected to one or more rechargeable battery assemblies disposed within the front clutch assembly 150, at least one accelerometer assembly 260 disposed within each of the plurality of electrode assemblies 250, the electrode assemblies 250 designed to vibrate.

FIG. 9B illustrates that the method for wearing a utility clutch for stimulating activity awareness to engage abdominal muscles for a user may further include 950 that the one or more containment members 155 are rendered into an open, closed, or partially open state by slidably moving one or more tab members 175 along the two flexible strips of metal or plastic with interlocking projections 156.

FIG. 9B further illustrates that the method for wearing a utility clutch for stimulating activity awareness to engage abdominal muscles for a user may involve 960 activating the Bluetooth GPS 183 and safety alert unit 184 on the front clutch assembly 150 to send data to apps via at least one or more of the Bluetooth GPS 183, the accelerometers 260, and the at least one Safety alert trackers 262.

FIG. 9B further illustrates that the method for wearing a utility clutch for stimulating activity awareness to engage abdominal muscles for a user may involve 970 connecting the at least one or more of the Bluetooth GPS 183, the at least one accelerometer 260, and the at least one Safety alert trackers 262 to at least one or more of a healthcare provider and a care support provider and sending and receiving data from the at least one electrode assembly 250.

FIG. 9B further illustrates that the method for wearing a utility clutch for stimulating activity awareness to engage abdominal muscles for a user may involve 980 utilizing using a live or recorded video training designed to encourage and motivate movement.

FIG. 9B further illustrates that the method for wearing a utility clutch for stimulating activity awareness to engage abdominal muscles for a user may include 990 utilizing at least one charging port member 264 disposed within the utility clutch to charge at least one or more of the at least one rechargeable battery members 153, the Bluetooth GPS 183, and the at least one at least one safety alert tracker 262.

The following patents are incorporated by reference in their entireties: U.S. Pat. Nos. 20100217096A1, 20170055834A1, and 20190336020A1.

While the inventive concept has been described above in terms of specific embodiments, it is to be understood that the inventive concept is not limited to these disclosed embodiments. Upon reading the teachings of this disclosure, many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A utility clutch for stimulating activity awareness to engage abdominal muscles for a user comprising:
a belt and buckle assembly with a belt member adapted to circumscribe the user's waist and a buckle assembly adapted to be adjustably fastened so as to position the buckle assembly substantially to the right or left side of the user's waist;
a front clutch assembly horizontally coupled to an outside face of a vertically widened portion of the belt member and facing outward from the user, the front clutch assembly having one or more containment members;
a back pouch assembly slidably coupled to the belt member of the belt and buckle assembly, a sealable back pouch containment member adapted to fold over the belt member so as to be interior to an inside face of the belt member and disposed substantially against the user's lower back, the back pouch containment member adapted to contain one or more cold pack or heat pack members;
one or more rechargeable battery assemblies disposed within the front clutch assembly; and
a plurality of electrode assemblies disposed on an inside face of the vertically widened belt portion, electronically connected to the battery assembly, at least one accelerometer assembly disposed within each of the plurality of electrode assemblies, the electrode assemblies adapted to vibrate upon detecting movement or non-movement of abdominal muscles.

2. The utility clutch for stimulating activity awareness to engage abdominal muscles for a user of claim 1 wherein the one or more containment members are adapted to be in an open, closed, or partially open state by slidably moving one or more tab members along two flexible strips of metal or plastic with interlocking projections.

3. The utility clutch for stimulating activity awareness to engage abdominal muscles for a user of claim 1 wherein the one or more containment members include a smartphone containment member and a pocket member containing a plurality of sealable or non-sealable compartments.

4. The utility clutch for stimulating activity awareness to engage abdominal muscles for a user of claim 1 wherein a plurality of loop members is outwardly disposed on an outside face of the belt member.

5. The utility clutch for stimulating activity awareness to engage abdominal muscles for a user of claim 1 wherein a plurality of substantially planar magnet members is horizontally disposed on an inside face of the back pouch containment member, the magnet members substantially covered by a fabric portion of the back pouch containment member and adapted to rest substantially against a user's lower back.

6. The utility clutch for stimulating activity awareness to engage abdominal muscles for a user of claim 1 wherein a Bluetooth GPS and at least one safety alert tracker are disposed on the front clutch assembly.

7. The utility clutch for stimulating activity awareness to engage abdominal muscles for a user of claim 1 wherein one or more electrode assemblies are adapted to record movement and send data to applet via at least one or more of Bluetooth GPS, accelerometers, and the at least one safety alert tracker.

8. The utility clutch for stimulating activity awareness to engage abdominal muscles for a user of claim 1 wherein at least one charging port member is disposed within the utility clutch electrically coupled to at least one or more of the rechargeable battery members, the Bluetooth GPS, and the at least one safety alert tracker.

9. A utility clutch for stimulating activity awareness to engage abdominal muscles for a user comprising:
a belt and buckle assembly with a belt member adapted to circumscribe the user's waist and a buckle assembly adapted to be adjustably fastened so as to position the buckle assembly substantially to the right or left side of the user's waist;
a front clutch assembly horizontally coupled to an outside face of a vertically widened portion of the belt member and facing outward from the user, the front clutch assembly having an inner containment member and an outer containment member, the inner containment member being wider and taller than the outer containment member and having a plurality of interior pocket members therein;
the one or more containment members adapted to be in an open, closed, or partially open state by slidably moving one or more of a left tab member and a right tab member along two flexible strips of metal or plastic with interlocking projections;
a back pouch assembly coupled to the belt member of the belt and buckle assembly, a sealable back pouch containment member adapted to fold over the belt member so as to be interior to an inside face of the belt member and disposed substantially against the user's lower back, the back pouch containment member adapted to contain one or more cold pack or heat pack members;
one or more rechargeable battery assemblies disposed within the front clutch assembly; and
at least six electrode assemblies disposed on an inside face of the vertically widened belt portion arranged so as to have at least one upper row and at least one lower row of electrode assemblies, the electrode assemblies electronically connected to the battery assembly, at least one accelerometer assembly disposed within each of the at least six of the electrode assemblies, the electrode assemblies adapted to vibrate upon detecting abdominal muscle movement, including lack of movement.

10. The utility clutch for stimulating activity awareness to engage abdominal muscles for a user of claim 9 wherein at least three pocket members are disposed within the interior containment member, at least one of the at least three pocket members sealable via two flexible strips of metal or plastic with interlocking projections, the pocket members further adapted to be viewable from the top, and wherein the outer containment member is further adapted to contain a smartphone.

11. The utility clutch for stimulating activity awareness to engage abdominal muscles for a user of claim 9 wherein five or more substantially planar magnet members are horizontally disposed on an inside face of the back pouch containment member, the magnet members substantially covered by a fabric portion of the back pouch containment member and adapted to rest substantially against a user's lower back, the back pouch member further adapted to contain at least one hot pack member or cold pack member.

12. The utility clutch for stimulating activity awareness to engage abdominal muscles for a user of claim 9 wherein a Bluetooth GPS and a safety alert tracker are disposed on the front clutch assembly.

13. The utility clutch for stimulating activity awareness to engage abdominal muscles for a user of claim 9 wherein one or more electrode assemblies are adapted to record movement and send data to apps via at least one or more of Bluetooth GPS, accelerometers, and at least one safety alert tracker.

14. The utility clutch for stimulating activity awareness to engage abdominal muscles for a user of claim 9 wherein at least one charging port member is disposed within the utility clutch electrically coupled to at least one or more of the rechargeable battery members, the Bluetooth GPS, and the at least one safety alert tracker, the rechargeable battery member further being a lithium-based rechargeable battery member.

15. A method for wearing a utility clutch for stimulating activity awareness to engage abdominal muscles for a user, the method comprising:
adjusting and fastening a belt and buckle assembly with a belt member adapted to circumscribe the user's waist and a buckle assembly so as to circumscribe the user's waist with the buckle assembly positioned substantially to the right or left side of the user's waist;
inserting at one of at least one or more of at least one cold pack and at least one heat pack member into the back pouch containment member;
further positioning a front clutch assembly horizontally across the user's waist, the front clutch assembly horizontally coupled to an outside face of a vertically widened portion of the belt member to face outward from the user, the front clutch assembly having one or more containment members;
further positioning a sealable back pouch containment member coupled to the belt member of the belt and buckle assembly so it is folded over the belt member so as to be interior to an inside face of the belt member and disposed substantially against the user's lower back; and
having one or more electrode assemblies vibrate upon detecting abdominal muscle movement, including lack of movement, via a plurality of electrode assemblies disposed on an inside face of the vertically widened belt portion electronically connected to one or more rechargeable battery assemblies disposed within the front clutch assembly, at least one accelerometer assembly disposed within each of the plurality of electrode assemblies, the electrode assemblies adapted to vibrate.

16. The method for wearing a utility clutch for stimulating activity awareness to engage abdominal muscles for a user of claim 15 wherein the one or more containment members are rendered into an open, closed, or partially open state by slidably moving one or more tab members along two flexible strips of metal or plastic with interlocking projections.

17. The method for wearing a utility clutch for stimulating activity awareness to engage abdominal muscles for a user of claim 15 wherein a Bluetooth GPS and safety alert tracker on the front clutch assembly are activated to send data to apps via at least one or more of the Bluetooth GPS, an at least one accelerometer, and an at least one safety alert tracker.

18. The method for wearing a utility clutch for stimulating activity awareness to engage abdominal muscles for a user of claim 15 wherein the at least one or more of the Bluetooth GPS, the at least one accelerometer, and the at least one safety alert tracker are connected to at least one or more of a healthcare provider and a care support provider and send and receive data from the at least one electrode.

19. The method for wearing a utility clutch for stimulating activity awareness to engage abdominal muscles for a user of claim 15 further including live or recorded video training adapted to encourage and motivate movement.

20. The method for wearing a utility clutch for stimulating activity awareness to engage abdominal muscles for a user of claim 15 wherein at least one charging port member disposed within the utility clutch charges at least one or more of the rechargeable battery members, the Bluetooth GPS, and the safety alert tracker.
